(19) United States
(12) Patent Application Publication
(10) Pub. No.: US 2010/0009014 A1
(43) Pub. Date: Jan. 14, 2010

(54) Use of composition for manufacture of medicant and method for inhibiting formation of body fat

(75) Inventors: Frank Chiahung Mao, Taichung City (TW); Wen-Ying Chen, Longjing Township (TW); Chi-Ching Sun, Hsinchu City (TW); Chia-Hsin Liu, Miaoli City (TW)

Correspondence Address: BACON & THOMAS, PLLC, 625 SLATERS LANE, FOURTH FLOOR, ALEXANDRIA, VA, 22314-1176, US

(73) Assignee: Maxluck Biotechnology Corp., Taipei City (TW)

(21) Appl. No.: 12/457,861

(22) Filed: Jun. 24, 2009

(30) Foreign Application Priority Data
Jul. 14, 2008 (TW) 097126609

(51) Int. Cl. A61K 38/40; A61K 33/24; A61P 3/00

(52) U.S. Cl. 424/655; 514/12

(57) ABSTRACT

The present invention relates to use of a composition for the manufacture of a medicament and a method for inhibiting the formation of body fat. The composition includes lactoferrin and a trivalent chromium compound. Herein, the chromium compound is selected from the group consisting of chromium (III) chloride hexahydrate, chromium (III) chloride, chromium (III) acetate, chromium (III) sulfate, chromium picolinate, chromium nicotinate, chromium GTF, chromium yeast extract, other inorganic salts of trivalent chromium, other organic salts of trivalent chromium, and combinations thereof. Accordingly, the composition of the present invention can assist in transmitting glucose from cells to muscle tissues and thereby reducing the storage of fat converted from glucose to achieve the purpose of controlling body weight.

### BACKGROUND OF THE INVENTION

1. Field of the Invention

The present invention relates to use of a composition for the manufacture of a medicament and a method for inhibiting the formation of body fat and, more particularly, to use of a composition containing trivalent chromium lactoferrin for the manufacture of a medicament and a method for inhibiting the formation of body fat.

2. Description of the Related Art

The obese population keeps increasing due to the impact of fast food culture and the tendency towards light-active lifestyles resulting from the development of science and technology, computerization and mechanization. Obesity can increase the risk for diabetes, cardiovascular disease, hypertension and so on, and thereby badly influences quality of personal life, increases social medical burden, and reduces the national competition. Therefore, in order to ensure healthy, it is really important for the modern humans to study how to control body weight.

Development of obesity results from adipocyte hyperplasia, adipocyte hypertrophy or adipocyte hyperplasia as well as adipocyte hypertrophy. Each adipocyte contains triglycerides therein. The rise in the triglyceride level results in the increase in adipocyte size and thereby leads to obesity. On the contrary, burning triglycerides can reduce the size of adipocytes to thereby achieve slimming. In a normal condition, the number of adipocytes stops increasing after the age of puberty. Thereby, for adults, gaining weight is caused by adipocyte hypertrophy due to storing unnecessary fat in adipocytes.

Since obesity results from unnecessary calorie being changed into fat and stored in body, the weight loss medicaments used in current medical fraternity generally are: (1) those for reducing appetite, such as sibutramine hydrochloride monohydrate; or (2) those for inhibiting nutrition absorption, such as orlistat. However, when those for reducing appetite or inhibiting nutrition absorption are used, some adverse effects will occur. For example, the side effects of sibutramine hydrochloride monohydrate include headache, nausea, vertigo, thirsty and sleeplessness, while the side effects of orlistat include gastrointestinal disturbances.

Accordingly, the purpose of the present invention is to provide health products with the ability to control body weight and result in no side effects.

### SUMMARY OF THE INVENTION

In comparison to those traditional products for weight loss by, for example, reducing appetite or inhibiting nutrition absorption, the present invention provides a composition for inhibiting the formation of body fat by assisting in transmitting glucose from cells to muscle tissues and thereby reducing the storage of fat converted from glucose to achieve the purpose of controlling body weight.

To achieve the object, the present invention provides a composition for inhibiting the formation of body fat, comprising: (a) lactoferrin and (b) a trivalent chromium compound. In addition, the present invention provides use of the aforementioned composition for the manufacture of a medicament for inhibiting the formation of body fat to an acceptor. Also, the present invention further provides a method for inhibiting the formation of body fat in an acceptor, comprising: proving an effective amount of the aforementioned composition to the acceptor.

The lactoferrin in the compostion of the present invention is not particularly restricted, and can come from cow lactoferrin, goat lactoferrin, unpurified cow milk, unpurified goat milk or a combination thereof. Because lactoferrin mainly exists in the whey of the milk, the lactoferrin in the composition of the present invention can also be completely or partly replaced with whey protein products or buttermilk powder.

The trivalent chromium compound in the composition of the present invention is not particularly restricted, either. The trivalent chromium compounds can be inorganic salts of trivalent chromium, organic salts of trivalent chromium or a combination thereof.

Inorganic salts of trivalent chromium include, for example, chromium (III) chloride hexahydrate, chromium (III) chloride and chromium (III) sulfate.

Organic salts of trivalent chromium include, for example, chromium (III) acetate, chromium picolinate, chromium nicotinate, amino acid chelated chromium, chromium GTF, chromium yeast extract (such as chromium brewer's yeast extract), and chromium yeast.

Preferably, the trivalent chromium compound is selected from the group consisting of chromium (III) chloride hexahydrate, chromium (III) chloride, chromium (III) acetate, chromium (III) sulfate, chromium picolinate, chromium nicotinate, chromium GTF, chromium yeast extract and a combination thereof.

In general, the molar ratio of lactoferrin to the trivalent chromium compound in the composition of the present invention is not particularly restricted. Preferably, the molar ratio of the trivalent chromium compound to lactoferrin ranges from 1:0.001 to 1:10. More preferably, the molar ratio of the trivalent chromium compound to lactoferrin ranges from 1:0.01 to 1:1.

The composition of the present invention can be used to form a medicament. Also, it can be added into a dairy product, and thereby form a dairy product containing trivalent chromium compound and lactoferrin, i.e., form a food or nutriment. The dairy product can be selected from the group consisting of the fresh milk of mammals, long-life milk, concentrated milk, cheese and milk powder.

In the composition of the present invention, the lactoferrin is a glycoprotein that is capable of binding with metal ions. Each lactoferrin molecule can be bound with two trivalent chromium ions to form a trivalent chromium-lactoferrin complex. In comparison to the low absorption rate of inorganic chromium and organic chromium (the absorption rate of inorganic chromium only ranges from 0.4% to 3%), the trivalent chromium-lactoferrin complex in the composition of the present invention can be more efficiently absorbed and utilized by the human body.

Accordingly, the composition containing trivalent chromium lactoferrin of the present invention can be taken by obese individuals. Taking regularly the composition containing trivalent chromium lactoferrin of the present invention not only can replenish the organic chromium efficiently, but also can assist in transmitting glucose from cells to muscle tissues and thereby reducing the storage of fat converted from glucose to favor the burning of fat and the construction and repair of muscle, so that the body fat and body weight can be controlled well. Also, the composition containing trivalent chromium lactoferrin of the present invention can prevent obese individuals from hyperleptinemia caused by leptin resistance.

### DETAILED DESCRIPTION OF THE PREFERRED EMBODIMENTS

The composition of the present invention can be formed by mixing the powder of lactoferrin with the powder of trivalent chromium compound. Moreover, water can also be added into the mixture of lactoferrin and the trivalent chromium compound to form a mixed solution. The mixed solution can be heated properly so that the mixing can be done adequately. The heating temperature ranges around 37° C. to 95° C., and preferably ranges from 50° C. to 80° C. The well-mixed solution can be then spray-dried to form the composition containing trivalent chromium lactoferrin of the present invention.

The raw material of the trivalent chromium compound used in the present invention can be inorganic salts or organic salts, such as chromium (III) chloride hexahydrate, chromium (III) chloride, chromium (III) acetate, chromium (III) sulfate, chromium picolinate, chromium nicotinate, chromium GTF, chromium yeast extract or chromium yeast.

Lactoferrin could come from the solution or dry powder of lactoferrin, unpurified cow milk or unpurified goat milk. Because lactoferrin mainly exists in the whey of the milk, the present invention can also use an unpurified whey protein product or buttermilk powder.

The following detailed description is given by way of example and not intended to limit the invention solely to the embodiments described herein.

### EXAMPLE 1

Mix 3.0 g of lactoferrin powder with 0.5 g of chromium (III) chloride hexahydrate and 1 liter of water to form a solution. The resultant solution is spray-dried and then mixed with 196 g of buttermilk powder and 100 g of whey protein to form the composition containing trivalent chromium lactoferrin of the present invention.

### EXAMPLE 2

Mix 60 g of lactoferrin powder and 400 g of whey protein with 1 g of chromium (III) chloride hexahydrate and water to form a solution and heat the solution up to 50° C. The resultant solution is mixed with 200 kg of buttermilk powder and spray-dried to form the composition containing trivalent chromium lactoferrin of the present invention.

### EXAMPLE 3

Mix 3 g of lactoferrin powder and 30 g of whey protein with 154.5 g of chromium (III) chloride hexahydrate and water to form a solution and heat the solution up to 50° C. The resultant solution is mixed with 50 kg of buttermilk powder and 25 kg of whey protein and spray-dried to form the composition containing trivalent chromium lactoferrin of the present invention.

### TEST EXAMPLE 1

The dairy product obtained from Example 1 is mixed into a mouse diet (Diet Induced Obesity Rodent Purified Diet w/60% Energy From Fat, TestDiet). The C57BL/6JNarl mice are randomly divided into two groups. The mice in the experimental group are fed with the mouse diet containing the dairy product (0.12 g/kg BW/day, containing 40 μg/kg BW/day of trivalent chromium), while those in the control group are fed with the mouse diet containing no dairy product. The C57BL/6JNarl mice aged 8 weeks are fed for 8 weeks and the body weights of the tested mice are recorded per week, as shown in Table 1.

The body weights of the mice in the experimental group (supplied with the dairy product) are significantly less than those in the control group (supplied with no dairy product) during the period from the first week to the eighth week. These results suggest that the body weights of the mice in the experimental group are controlled well.

TABLE 1
Changes of body weight (unit: g)

| | Control group | Experimental group |
|---|---|---|
| Week | (N = 6) | (N = 6) |
| 0 | 20.6 ± 0.8 | 20.1 ± 0.48 |
| 1 | 23.9 ± 0.82 | 22.6 ± 1.07* |
| 2 | 26.2 ± 1.36 | 24.5 ± 1.17* |
| 3 | 27.6 ± 1.58 | 25.0 ± 2.07* |
| 4 | 30.5 ± 1.62 | 27.2 ± 2.10* |
| 5 | 32.7 ± 2.01 | 28.4 ± 2.31** |
| 6 | 34.7 ± 2.26 | 30.4 ± 2.29** |
| 7 | 35.6 ± 2.62 | 30.9 ± 2.19** |
| 8 | 37.4 ± 3.11 | 32.1 ± 2.59** |

*p < 0.05, significant difference vs. control group.
**p < 0.01, significant difference vs. control group.
N means the number of mice.

### TEST EXAMPLE 2

The dairy product obtained from Example 1 is mixed into a mouse diet (Diet Induced Obesity Rodent Purified Diet w/60% Energy From Fat, TestDiet). The C57BL/6JNarl mice are randomly divided into two groups. The mice in the experimental group are fed with the mouse diet containing the dairy product (0.12 g/kg BW/day, containing 40 μg/kg BW/day of trivalent chromium), while those in the control group are fed with the mouse diet containing no dairy product. The C57BL/6JNarl mice aged 8 weeks are fed for 8 weeks and then sacrificed. The change of body fat is estimated by observing the weights of Epididymal fat and perirenal fat, as shown in Table 2. Table 2 shows that the weights of Epididymal fat and perirenal fat in the experimental group are significantly reduced, and thereby it can be recognized that the dairy product provides the efficiency for inhibiting the formation of body fat.

TABLE 2

| | Control group | Experimental group |
|---|---|---|
| | (N = 6) | (N = 6) |
| Epididymal fat (g) | 2.341 ± 0.329 | 1.724 ± 0.264** |
| Perirenal fat (g) | 0.955 ± 0.08 | 0.661 ± 0.112*** |

*p < 0.05, significant difference vs. control group.
**p < 0.01, significant difference vs. control group.
N means the number of mice.

### TEST EXAMPLE 3

The dairy product obtained from Example 1 is mixed into a mouse diet (Diet Induced Obesity Rodent Purified Diet w/60% Energy From Fat, TestDiet). The C57BL/6JNarl mice are randomly divided into two groups. The mice in the experimental group are fed with the mouse diet containing the dairy product (0.12 g/kg BW/day, containing 40 μg/kg BW/day of trivalent chromium), while those in the control group are fed with the mouse diet containing no dairy product. The C57BL/6JNarl mice aged 8 weeks are fed for 8 weeks and then sacrificed to observe the blood level of leptin, as shown in Table 3. Table 3 shows that the blood level of leptin in the experimental group is significantly reduced, and thereby it can be recognized that the dairy product has the ability to improve hyperleptinemia.

TABLE 3

| | Control group | Experimental group |
|---|---|---|
| | (N = 6) | (N = 6) |
| Blood Leptin (ng/ml) | 28.1 ± 5.7 | 12.8 ± 1.7*** |

***p < 0.001, significant difference vs. control group.
N means the number of mice.

### TEST EXAMPLE 4

The dairy product obtained from Example 1 is mixed into a mouse diet (Diet Induced Obesity Rodent Purified Diet w/60% Energy From Fat, TestDiet). The C57BL/6JNarl mice are randomly divided into two groups. The mice in the experimental group are fed with the mouse diet containing the dairy product (0.12 g/kg BW/day, containing 40 μg/kg BW/day of trivalent chromium), while those in the control group are fed with the mouse diet containing no dairy product. The C57BL/6JNarl mice aged 8 weeks are fed for 8 weeks and then sacrificed to harvest partial Epididymal fat. Then, the Epididymal fat is fixed with 10% neutral formalin solution and embedded with paraffin wax. Serial sections are cut from each specimen and stained with hematoxylin and eosin (H&E). After staining, the sections are analyzed under ×100 microscopy. Each section is observed under five various fields of view to select fifty adipocytes and the diameters of the adipocytes are measured, in which the average value of the diameters means the adipocyte size of mice. The results suggest that the adipocytes of the mice in the control group are full of fat droplets and thus larger than those in the experimental group. However, after the supplement of the dairy product, the size of adipocytes in the experimental group is significantly reduced, as shown in Table 4.

TABLE 4
(unit: μm)

| | Control group | Experimental group |
|---|---|---|
| No. | (N = 6) | (N = 6) |
| 1 | 544 | 298 |
| 2 | 517 | 385 |
| 3 | 404 | 340 |
| 4 | 511 | 360 |
| 5 | 410 | 349 |
| 6 | 365 | 332 |
| Average | 459 ± 67.7 | 344 ± 26.6** |

**p < 0.01, significant difference vs. control group.
N means the number of mice.

From Tables 1 to 4, it can be proved that the dairy product of the present invention can efficiently control the body weight and inhibit the formation of body fat. For mice, the supplement of about 40 μg/kg BW/day Cr³⁺ contained in the trivalent chromium lactoferrin composition can achieve the efficiency of inhibiting the formation of body fat and controlling the body weight. Accordingly, based on the ratio in metabolic rates of acceptors (the metabolic rate of mice is ten times larger than that of human), it can be estimated that, for human, the supplement of about 4 μg/kg BW/day Cr³⁺ contained in the trivalent chromium lactoferrin composition can achieve the efficiency of inhibiting the formation of body fat and controlling the body weight.

### TEST EXAMPLE 5

The C57BL/6JNarl mice (N=70) are fed with a mouse diet (high-fat Rodent TestDiet, PMI Nutrition International Inc., MO, U.S.A.; 67% of calories provided by fat). The C57BL/6JNarl mice are randomly divided into seven groups and there are ten mice in each group. In one experimental group, the mice are fed with the mouse diet mixed with lactoferrin (NZMP lactoferrin, New Zealand, low dose: 40 mg/kg BW/day, high dose: 80 mg/kg BW/day). In another experimental group, the mice are fed with the mouse diet mixed with trivalent chromium (chromium (III) chloride hexahydrate, low dose: 40 μg/kg BW/day Cr³⁺, high dose: 80 μg/kg BW/day Cr³⁺). In yet another experimental group, the mice are fed with the mouse diet mixed with a lactoferrin/trivalent chromium composition (low dose: lactoferrin of 40 mg/kg BW/day with Cr³⁺ of 40 μg/kg BW/day, high dose: lactoferrin of 80 mg/kg BW/day with Cr³⁺ of 80 μg/kg BW/day). In the control group, the mice are fed with the mouse diet containing no other additive. The C57BL/6JNarl mice aged 8 weeks are fed for 7 weeks and then sacrificed to measure the weight of Epididymal fat and body weight, as shown in Table 5.

TABLE 5

| | | Epididymal fat weight | Body weight |
|---|---|---|---|
| | | (g) | (g) |
| Control group | | 0.96 ± 0.15 | 28.1 ± 1.51 |
| Lactoferrin | Low dose | 0.72 ± 0.28 | 27.7 ± 2.19 |
| | High dose | 0.82 ± 0.2 | 27.9 ± 1.37 |
| Trivalent Chromium | Low dose | 0.78 ± 0.16 | 27.8 ± 1.33 |
| | High dose | 0.82 ± 0.2 | 27.7 ± 1.35 |
| Lactoferrin + | Low dose | 0.60 ± 0.10*** | 26.7 ± 1.19* |
| Trivalent Chromium | High dose | 0.58 ± 0.12*** | 26.8 ± 0.98* |

*p < 0.05, significant difference vs. control group.
***p < 0.001, significant difference vs. control group.

Table 5 shows that the Epididymal fat weight and body weight of mice in the control group are larger. However, the Epididymal fat weight and body weight of mice are significantly reduced after the supplement of the lactoferrin/trivalent chromium composition, while the supplement of single lactoferrin or trivalent chromium cannot achieve significant efficiency. Accordingly, it can be known that the lactoferrin/trivalent chromium composition provides more significant efficiency in comparison to single lactoferrin or trivalent chromium.

In conclusion, the composition containing trivalent chromium lactoferrin of the present invention can be taken by those in high risk group for obesity or a patient suffering from obesity to control body fat, body weight and the size of adipocytes, improve hyperlipidemia, and thereby achieve the purpose for controlling body weight.

Although the present invention has been explained in relation to its preferred embodiments, it is to be understood that many other possible modifications and variations can be made without departing from the scope of the invention as hereinafter claimed.

What is claimed is:

1. Use of a composition for the manufacture of a medicament for inhibiting the formation of body fat to an acceptor, wherein the composition comprises:
a lactoferrin; and
a trivalent chromium compound;
wherein the trivalent chromium compound is selected from the group consisting of chromium (III) chloride hexahydrate, chromium (III) chloride, chromium (III) acetate, chromium (III) sulfate, chromium picolinate, chromium nicotinate, chromium GTF, chromium yeast extract, other inorganic salts of trivalent chromium, other organic salts of trivalent chromium, and combinations thereof.

2. The use as claimed in claim 1, wherein the molar ratio of the trivalent chromium compound to the lactoferrin ranges from 1:0.001 to 1:10.

3. The use as claimed in claim 1, wherein the molar ratio of the trivalent chromium compound to the lactoferrin ranges from 1:0.01 to 1:1.

4. The use as claimed in claim 1, wherein the lactoferrin comes from unpurified milk or whey protein.

5. The use as claimed in claim 1, wherein the lactoferrin is selected from the group consisting of cow lactoferrin, goat lactoferrin, unpurified cow milk, unpurified goat milk, and combinations thereof.

6. The use as claimed in claim 1, wherein the trivalent chromium compound is selected from the group consisting of chromium (III) chloride hexahydrate, chromium (III) chloride, chromium (III) acetate, chromium (III) sulfate, chromium picolinate, chromium nicotinate, chromium GTF, chromium yeast extract, and combinations thereof.

7. The use as claimed in claim 1, wherein the composition serves as an additive of a dairy product, which is selected from the group consisting of fresh milk of mammals, long-life milk, concentrated milk, fermented milk, cheese, and milk powder.

8. A method for inhibiting the formation of body fat in an acceptor, comprising: administrating an effective amount of a composition for inhibiting the formation of body fat to the acceptor, wherein the composition comprises:
a lactoferrin; and
a trivalent chromium compound;
wherein the trivalent chromium compound is selected from the group consisting of chromium (III) chloride hexahydrate, chromium (III) chloride, chromium (III) acetate, chromium (III) sulfate, chromium picolinate, chromium nicotinate, chromium GTF, chromium yeast extract, other inorganic salts of trivalent chromium, other organic salts of trivalent chromium, and combinations thereof.

9. The method as claimed in claim 8, wherein the molar ratio of the trivalent chromium compound to the lactoferrin ranges from 1:0.001 to 1:10.

10. The method as claimed in claim 8, wherein the molar ratio of the trivalent chromium compound to the lactoferrin ranges from 1:0.01 to 1:1.

11. The method as claimed in claim 8, wherein the lactoferrin comes from unpurified milk or whey protein.

12. The method as claimed in claim 8, wherein the lactoferrin is selected from the group consisting of cow lactoferrin, goat lactoferrin, unpurified cow milk, unpurified goat milk, and combinations thereof.

13. The method as claimed in claim 8, wherein the trivalent chromium compound is selected from the group consisting of chromium (III) chloride hexahydrate, chromium (III) chloride, chromium (III) acetate, chromium (III) sulfate, chromium picolinate, chromium nicotinate, chromium GTF, chromium yeast extract, and combinations thereof.

14. The method as claimed in claim 8, wherein the composition serves as an additive of a dairy product, which is selected from a group consisting of fresh milk of mammals, long-life milk, concentrated milk, fermented milk, cheese, and milk powder.
